(19) United States
(12) Patent Application Publication
(10) Pub. No.: US 2021/0403338 A1
(43) Pub. Date: Dec. 30, 2021

(54) DUAL LIGHT-RESPONSIVE ZINC OXIDE AND PREPARATION METHOD THEREOF AS WELL AS PHOTOSENSITIVE COATING WITH ANTIBACTERIAL/OSTEOGENIC PROPERTIES

(71) Applicant: Nanchang University, Nanchang, Jiangxi (CN)

(72) Inventors: Lan LIAO, Nanchang, Jiangxi (CN); Xiaolei WANG, Nanchang, Jiangxi (CN); Siyu ZHAO, Nanchang, Jiangxi (CN)

(73) Assignee: Nanchang University, Nanchang, Jiangxi (CN)

(21) Appl. No.: 17/414,941

(22) PCT Filed: Jul. 10, 2020
(86) PCT No.: PCT/CN2020/101204

§ 371 Date: Jun. 16, 2021

(30) Foreign Application Priority Data
Jun. 24, 2020 (CN) 202010589977.7

(51) Int. Cl. C01G 9/02; A61L 27/30; A61L 27/54; A61L 27/06

(52) U.S. Cl. CPC C01G 9/02; C01P 2006/16; A61L 27/54; A61L 27/06; A61L 2300/102; A61L 2300/404; A61L 2300/412; A61L 2420/02; A61L 2430/12; C01P 2002/72; C01P 2002/82; C01P 2002/84; C01P 2004/03; C01P 2004/61; C01P 2006/12; C01P 2006/14; A61L 27/306

(57) ABSTRACT

Provided is a dual light-responsive zinc oxide, in the preparation process of zinc oxide, sodium citrate and hydroxypropyl methyl cellulose are added to control the morphology, photothermal conversion materials are added to make zinc oxide have photothermal conversion ability, and lignin is added to reduce the energy band gap of zinc oxide; and the hydrothermal products after lyophilization are carbonized by microwave irradiation so as to further reduce the energy band gap. The dual light-responsive zinc oxide has a Tremella-like fold structure, has dual response to yellow light and near-infrared light, has excellent adsorbability, antibacterial property and photothermal stability, and has photothermal conversion ability. The dual light-responsive zinc oxide coating has both antibacterial and osteogenic properties, which can efficiently improve the antibacterial and osteogenic capability of implants when being applied on the surface of the implants; and its special photosensitive property helps to realize the photocontrol working and on-demand action of the antibacterial and osteogenic functions of the implant.

### CROSS REFERENCE TO RELATED APPLICATION(S)

This patent application claims the benefit and priority of Chinese Patent Application No. 202010589977.7, entitled “Dual light-responsive zinc oxide and preparation method thereof as well as photosensitive coating with antibacterial/osteogenic properties”, filed to China National Intellectual Property Administration on Jun. 24, 2020, the disclosure of which is incorporated by reference herein in its entirety as part of the present application.

### TECHNICAL FIELD

The present disclosure belongs to the technical field of surgical implants, and specifically relates to a dual light-responsive zinc oxide and a preparation method thereof as well as a photosensitive coating with antibacterial/osteogenic properties.

### BACKGROUND ART

Oral implant surgery is one of the most common implant surgeries in humans. Meanwhile, the number of patients affected by peri-implant diseases (PIDs) is also increasing. Different from that of conventional totally-enclosed implant systems, the morbidity of oral peri-implant diseases is as high as about 40%. This is because the implant is partially exposed to oral microenvironment, which is likely to cause bacterial invasion and peri-implant mucositis (PIM); the further spread of inflammation may lead to gradual loss of the supporting bone around the implant, which further lead to the occurrence of peri-implantitis (PI). Bacterial infection is most closely related to PIDs of the implant, which may lead to weak bone bonding around the implant and the shedding of the implant, finally resulting in the failure of implantation. As such, more additional surgeries are needed for patients with loss of teeth to treat PIDs, and sometimes it's even necessary to undergo a secondary implantation to restore a missing tooth, thus causing a great impact on the body and mind of the patient.

Titanium-based implants are the most widely used implants in clinic. However, as an inert material, titanium itself has no antibacterial property, the osteointegration between titanium and the surrounding bone tissue is poor and the implant failure rate is high.

### SUMMARY

In view of this, the present disclosure provides a dual light-responsive zinc oxide and a preparation method thereof as well as a photosensitive coating with antibacterial/osteogenic properties. The dual light-responsive zinc oxide of the present disclosure has good adsorption capacity, can respond to yellow light and near-infrared light, and has good antibacterial property and photothermal conversion ability, so the coating prepared therefrom can significantly improve the antibacterial and osteogenic properties of the implant.

In order to realize the above objectives, the present disclosure provides the following technical schemes:

A preparation method of dual light-responsive zinc oxide, including the following steps:

(1) a soluble zinc salt, hexamethylenetetramine and water are mixed for a first hydrothermal reaction, and then the reaction material liquor is mixed with sodium citrate, hydroxypropyl methyl cellulose, photothermal conversion materials and lignin for a second hydrothermal reaction, to get hydrothermal products;

(2) the hydrothermal products are subjected to lyophilization and microwave irradiation successively to get the dual light-responsive zinc oxide.

Preferably, the soluble zinc salt is zinc nitrate.

Preferably, the photothermal conversion materials are one or more of activated carbon, gold rod and black phosphorus.

Preferably, the dosage ratio of the soluble zinc salt, hexamethylenetetramine, water, sodium citrate, hydroxypropyl methyl cellulose, photothermal conversion materials and lignin is (1.487-1.488) g:(0.350-0.352) g:(95-110) mL:(0.138-0.142) g:(0.100-0.120) g:(0.025-0.035) g:(0.995-0.115) g.

Preferably, for the first hydrothermal reaction, the temperature is 65-67° C., and the time is 14-16 min; for the second hydrothermal reaction, the temperature is 85-87° C., and the time is 10-12 h.

Preferably, for the microwave irradiation, the power is above 800 W, and the time is more than 15 min.

The present disclosure provides a dual light-responsive zinc oxide prepared by the method in the above scheme, the dual light-responsive zinc oxide has a Tremella-like microtopography; and the dual light-responsive zinc oxide can respond under the irradiation of yellow light and near-infrared light.

The present disclosure also provides a photosensitive coating with antibacterial/osteogenic properties, which is prepared from the dual light-responsive zinc oxide prepared in the above scheme.

Preferably, the coating is prepared by the following steps:

the dual light-responsive zinc oxide is dispersed in a solvent, the resulting suspension is coated on the surface of a substrate and dried to get the photosensitive coating with antibacterial/osteogenic properties; and the substrate is a surgical implant.

Preferably, the raw materials for preparing the coating further include type I collagen powder, and the mass ratio of the dual light-responsive zinc oxide to the type I collagen powder is 1:(1-5).

The present disclosure provides a preparation method of dual light-responsive zinc oxide, in which a soluble zinc salt, hexamethylenetetramine and water are mixed for a first hydrothermal reaction, and then the reaction material liquor is mixed with sodium citrate, hydroxypropyl methyl cellulose, photothermal conversion materials and lignin for a second hydrothermal reaction, to get hydrothermal products; the hydrothermal products are subjected to lyophilization and microwave irradiation successively to get the dual light-responsive zinc oxide. In the preparation process of the present disclosure, sodium citrate and hydroxypropyl methyl cellulose are added to control the morphology of zinc oxide, photothermal conversion materials are added to make the resulting zinc oxide have photothermal conversion ability, and lignin is added to reduce the energy band gap of zinc oxide; and after the completion of hydrothermal reactions, the resulting hydrothermal products are lyophilized and then carbonized by microwave irradiation so as to further reduce the energy band gap of zinc oxide, so that the material can respond to long-wavelength visible light (yellow light); meanwhile, the carbonization by microwave irradiation can also make zinc oxide have a more obvious Tremella-like fold structure, thereby improving its adsorptive capacity.

The present disclosure provides dual light-responsive zinc oxide prepared by the method in the above scheme. The dual light-responsive zinc oxide prepared in the present disclosure has a Tremella-like fold structure, has excellent adsorbability (being capable of adsorbing pigments, proteins and other substances), antibacterial property and photothermal stability, and has photothermal conversion ability. The dual light-responsive zinc oxide has photocatalytic effects under the irradiation of yellow light which can further improve the antibacterial ability, and has photothermal conversion ability under the irradiation of near-infrared light that makes it a good photothermal treating material.

The present disclosure also provides a photosensitive coating with antibacterial/osteogenic properties, which is prepared from the dual light-responsive zinc oxide in the above scheme. The coating of the present disclosures has both antibacterial and osteogenic properties, which can be activated under the irradiation of safe and gentle visible light (yellow light), thus having good antibacterial properties against Gram-positive bacteria and Gram-negative bacteria, especially having obvious bacteriostatic effect on specific oral bacteria. In addition, the coating shows good photothermal conversion ability under near-infrared (NIR) irradiation, and the moderate rise in temperature can efficiently promote the proliferation of bone mesenchymal stem cells (BMSCs) and the expression of osteogenic gene, and promote the bone bonding between the implant and the surrounding bone tissue. The coating of the present disclosure applied on the surface of the titanium-based implant can efficiently improve the antibacterial and osteogenic effects of the titanium-based implant, and its special photosensitive property helps to realize the photocontrol working and on-demand action of the antibacterial and osteogenic functions of the implant.

Furthermore, the raw materials for preparing the photosensitive coating of the present disclosure further include type I collagen powder, which has the effect of promoting osteogenesis and thus can further improve the osteogenic property of the coating.

### BRIEF DESCRIPTION OF THE DRAWINGS

FIG. 1 shows the scanning electron microscope photographs of ZnO and ZnO-Col-I, with a scale of 1 μm;

FIG. 2 shows the energy spectra of ZnO and ZC;

FIG. 3 shows the X-ray diffraction pattern of Ti and Ti—ZnO;

FIG. 4 shows the standard curve of bovine serum albumin in embodiment 3;

FIG. 5 is a chart showing the adsorption results of BSA by different concentrations of zinc oxide in embodiment 3;

FIG. 6 shows the ultra-violet absorption spectra of Rhodamine B solution under dark and light conditions respectively for 0 h, 12 h, 24 h, 36 h and 48 h as in embodiment 4;

FIG. 7 is a curve showing the changes of degradation rate of Rhodamine B under dark and light conditions respectively as in embodiment 4;

FIG. 8 is a diagram showing the temperature changes of different kinds of zinc oxide dispersion under light condition as in embodiment 5;

FIG. 9 shows a thermal imaging map of titanium samples through different treatments before and after NIR irradiation as in embodiment 5;

FIG. 10 shows the antibacterial ratio of Ti samples in embodiment 6 against S. aureus and the SEM view of the bacterium (the scale is 1 μm);

FIG. 11 shows the antibacterial ratio of Ti samples in embodiment 6 against E. coli and the SEM view of the bacterium (the scale is 1 μm);

FIG. 12 shows the antibacterial ratio of Ti samples in embodiment 6 against S. mutans and the SEM view of the bacterium (the scale is 1 μm);

FIG. 13 is a diagram showing the temperature changes of cells treated with different impregnating liquids as in embodiment 7 under NIR irradiation;

FIG. 14 shows the infrared thermogram of cells treated with different impregnating liquids as in embodiment 7 after continuous NIR irradiation for 10 min;

FIG. 15 shows the gene expression level of Runx2 and OCN of cells after being treated with different impregnating liquids as in embodiment 8;

FIG. 16 shows the testing results of ALP staining level of cells after being treated with different impregnating liquids as in embodiment 8, (the scale is 200 μm).

### DETAILED DESCRIPTION OF THE EMBODIMENTS

The present disclosure will be further illustrated below in combination with the embodiments and the attached drawings.

The present disclosure provides a preparation method of dual light-responsive zinc oxide, which includes the following steps:

(1) a soluble zinc salt, hexamethylenetetramine and water are mixed for a first hydrothermal reaction, and then the reaction material liquor is mixed with sodium citrate, hydroxypropyl methyl cellulose, photothermal conversion materials and lignin for a second hydrothermal reaction, to get hydrothermal products;

(2) the hydrothermal products are subjected to lyophilization and microwave irradiation successively to get the dual light-responsive zinc oxide.

Unless otherwise specified, the water used in the present disclosure is deionized water.

In the present disclosure, a soluble zinc salt, hexamethylenetetramine (HMT) and water are mixed for a first hydrothermal reaction. In the present disclosure, the soluble zinc salt is preferably zinc nitrate, specifically zinc nitrate hexahydrate; the water is preferably deionized water; for the first hydrothermal reaction, the temperature is preferably 65-67° C., more preferably 65-66° C., and the time is preferably 14-16 min, more preferably 15 min. In the present disclosure, it is preferable to dissolve the soluble zinc salt and hexamethylenetetramine in water and stir for 10 min to dissolve the soluble zinc salt and hexamethylenetetramine completely, and then go on to the first hydrothermal reaction; the stirring is preferably carried out under a sealed condition. During the first hydrothermal reaction, the soluble zinc salt provides Zn²⁺, and HMT releases OH⁻, Zn²⁺ and OH⁻ slowly during the reaction, and the resulting ions react under hydrothermal conditions to generate Zn(OH)₂ colloid that then further reacts to generate zinc oxide.

Upon the completion of the first hydrothermal reaction, the reaction material liquor is mixed with sodium citrate, hydroxypropyl methyl cellulose (HPMC), photothermal conversion materials and lignin for the second hydrothermal reaction to get hydrothermal products. In the present disclosure, the dosage ratio of the soluble zinc salt, hexamethylenetetramine, water, sodium citrate, hydroxypropyl methyl cellulose, photothermal conversion materials and lignin is preferably (1.487-1.488) g:(0.350-0.352) g:(95-110) mL:(0.138-0.142) g:(0.100-0.120) g:(0.025-0.035) g:(0.995-0.115) g, more preferably 1.4875 g:0.351 g:100 mL:0.14 g:0.1 g:0.025 g:0.1 g; the photothermal conversion materials are preferably one or more of activated carbon, gold rod and black phosphorus, more preferably activated carbon; the sodium citrate acts as the surfactant, and the hydroxypropyl methyl cellulose plays a molding role, both of them can control the morphology of zinc oxide; photothermal conversion materials enable zinc oxide to have the photothermal conversion ability; lignin has a good biocompatibility, which can reduce the energy band gap of zinc oxide to some extent.

In the present disclosure, for the second hydrothermal reaction, the temperature is preferably 85-87° C., more preferably 85-86° C., and the time is preferably 10-12 h, more preferably 10-11 h. During the second hydrothermal reaction, HMT in the solution continues to release OH⁻ slowly, which reacts with zinc ions to generate more zinc oxide; moreover, photothermal conversion materials, lignin and zinc oxide all act during the process so that the resulting zinc oxide has photothermal conversion ability, and its energy band gap can be reduced.

Upon the completion of the second hydrothermal reaction, it is preferable to filter the reaction material liquor, and then wash with anhydrous ethyl alcohol and water successively to get the hydrothermal products; it is preferable to wash with anhydrous ethyl alcohol and water for 2 times, the anhydrous ethyl alcohol washing and water washing are preferably centrifugal washing, the rotating speed for the centrifugal washing is preferably 7000 rpm, and the time for single washing is preferably 15 min.

After obtaining the hydrothermal products, they are subjected to lyophilization and microwave irradiation successively to get the dual light-responsive zinc oxide. In the present disclosure, the hydrothermal products are preferably lyophilized in vacuum after being pre-frozen at −80° C.; the time for vacuum lyophilization is preferably 12 h; for the microwave irradiation, the power is preferably above 800 W, more preferably 800-850 W, and the time is preferably more than 15 min, specifically 15-20 min.

The present disclosure provides a dual light-responsive zinc oxide prepared by the method in the above scheme. The dual light-responsive zinc oxide of the present disclosure has a Tremella-like microtopography, and is made up of fold lamellar structures stacked on the top of each other. The particle size of the dual light-responsive zinc oxide is about 2 μm, and its energy band gap is 2.125 eV (thus being capable of responding to yellow light). The dual light-responsive zinc oxide can respond under the irradiation of yellow light and near-infrared light. In specific embodiments of the present disclosure, it can respond specifically at 597-577 nm (yellow light) and 808 nm (near-infrared light). The dual light-responsive zinc oxide of the present disclosure has excellent adsorbability (being capable of adsorbing proteins, pigments, and other substances), and has good antibacterial property and photothermal conversion ability.

The present disclosure also provides a photosensitive coating with antibacterial/osteogenic properties, which is prepared from the dual light-responsive zinc oxide in the above scheme. The coating of the present disclosure has good antibacterial property that can be further improved because of its photocatalytic performance under yellow light, and it has photothermal conversion ability under near-infrared light, so the rise of temperature helps to promote osteogenesis. The special photosensitive property of the coating provided in the present disclosures helps to realize the photocontrol working and on-demand action of the antibacterial and osteogenic functions of the implant. For example, in oral implantation, after implant placement, the sites of oral contact (cuff) are likely to cause breeding of bacteria, which then spread down to the periodontal area. When the surface of the implant contains the coating of the present disclosure, a yellow-light toothbrush can be used to improve the antibacterial property of the implant due to the photocatalytic effect of the yellow light. Further for example, after the implantation of an implant provided with the photosensitive coating of the present disclosure, near-infrared light irradiation (i.e., thermal therapy) is given to promote bone bonding.

In the present disclosure, the coating is prepared preferably by the following steps:

The dual light-responsive zinc oxide is dispersed in a solvent, the resulting suspension is coated on the surface of a substrate and dried to get the photosensitive coating with antibacterial/osteogenic properties.

In the present disclosure, the substrate is preferably a surgical implant, and the surgical implant may be specifically made of pure titanium, titanium alloy, titanium-zirconium alloy and the like.

In the present disclosure, the solvent is preferably water, normal saline or PBS buffer. The dispersion is preferably carried out with stirring, the rotating speed for stirring is preferably 75 rpm, and the time is preferably 2 h. The concentration of the dual light-responsive zinc oxide in the suspension is preferably 200 μg/mL. The present disclosure has no special requirement on the coating amount of the suspension, as long as covering the surface of the substrate fully and uniformly. The drying temperature is preferably room temperature. The present disclosure has no specific limitation on the drying time, and it is advisable to dry thoroughly.

In the present disclosure, the raw materials for preparing the coating preferably also include type I collagen powder (Col-I). The mass ratio of the dual light-responsive zinc oxide to type I collagen powder is preferably 1:(1-5), and more preferably 1:(2-3). When the raw materials for preparing the coating include type I collagen powder, the type I collagen powder and the dual light-responsive zinc oxide are co-dispersed in a solvent, other conditions are the same as those in the above scheme and will not be repeated here. Col-I is the main organic substrate of natural bone, which participates in regulating the development, differentiation activity and bone remodeling of osteoblasts, and plays an important role in tissue repair and regeneration. Col-I is added for the preparation of the coating in the present disclosure, which can be adsorbed in the fold structure of zinc oxide, thus further improving the osteogenic capability of the coating.

The technical schemes of the present disclosure will be described clearly and completely below in combination with the embodiments of the present disclosure.

Embodiment 1

50 mmol (1.4875 g) Zn(NO₃)₂.6H₂O, 25 mmol (0.3505 g) HMT were dissolved in 100 mL deionized water, sealed and stirred for 10 min. After heating in a water bath at 65° C. for 15 min, 0.14 g Na₃C₆H₅O₇, 0.1 g HPMC, 0.025 g activated carbon and 0.1 g lignin were added, while maintaining the water bath at 85° C. for 10 h. They were washed with anhydrous ethyl alcohol for 2 times, and washed with deionized water for 2 times, both of which were centrifugal washing at a rotating speed of 7000 rpm, and the time of single washing was 15 min. After then, they were pre-frozen at −80° C. and then lyophilized in vacuum for 12 h. The resulting products were subjected to microwave irradiation at a power of 800 W for 15 min to get the dual light-responsive ZnO powder. The ZnO prepared in embodiment 1 were used in subsequent experiments.

Embodiment 2

1. Preparation of Ti-ZC: The ZnO powder prepared in embodiment 1 and type I collagen powder were added into phosphate buffered saline (PBS buffer) at a mass ratio of 1:1, and stirred at 75 rpm for 2 h to get a suspension, in which the concentration of ZnO powder was 200 μg/mL. The suspension was dropwise added onto the surface of titanium specimen (titanium sheets with a diameter of 10 mm and a thickness of 1 mm), and dried at normal temperature. The resulting titanium samples with coating were marked as Ti-ZC.

2. Preparation of Ti—ZnO: The same as in 1, except that no type I collagen powder was added and the concentration of ZnO in the suspension was 200 μg/mL. The resulting samples were marked as Ti—ZnO.

3. Preparation of ZnO-Col-I: By using PBS buffer, the same concentration (200 μg/ml) of ZnO and Col-I were mixed and shaken at 75-80 rpm in a shaker for 2-3 h, to prepare a ZnO-Col-I suspension. The resulting samples were marked as ZnO-Col-I, subsequently referred as ZC for short.

Characterization:

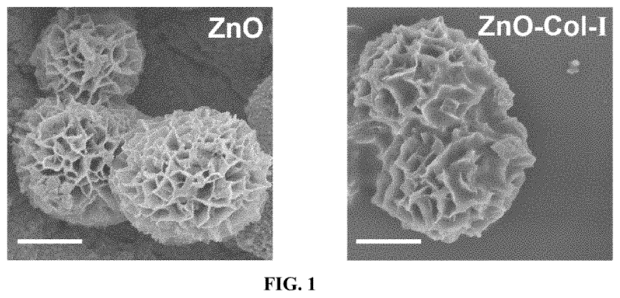

A scanning electron microscope was used to observe the morphology of ZnO and ZnO-Col-I, with the results shown in FIG. 1. FIG. 1 shows the scanning electron microscope photographs of ZnO and ZnO-Col-I, with a scale of 1 μm. It can be seen from FIG. 1 that, ZnO has a Tremella-like morphology and is made up of fold lamella stacked on the top of each other; and it can be seen from the scanning electron microscope photograph of ZnO-Col-I that Col-I was attached to the fold structure of ZnO.

A particle size and potential analyzer (Zeta-sizer Nano ZS90, Malvern, UK) was used to determine the size of ZnO, with the results showing that the particle size of ZnO was about 2 μm.

The energy band gap of ZnO was tested by an ultraviolet absorption method, with the results showing that the energy band gap of ZnO was 2.125 eV, and ZnO with such an energy band gap could be stimulated by yellow light.

A specific surface area and porosimetry analyzer (JW-BK132F) was used to measure the pore volume-pore size distribution of ZnO, with the results showing that the specific surface area of ZnO was 49.857 m²/g, the pore volume was 0.219 cm³/g, and the average pore size was 16.207 nm.

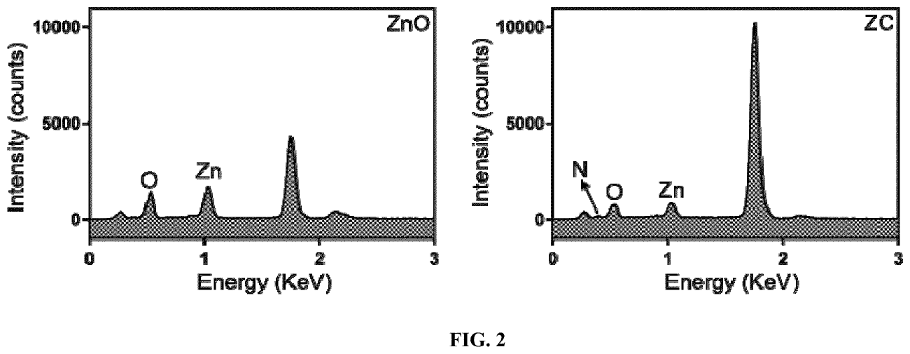

An energy dispersive spectrometer (EDS, Zeiss/Sigma 300, Japan) was used to characterize the chemical components of ZnO and ZC (i.e., ZnO-Col-I), with the results shown in FIG. 2. It can be seen from FIG. 2 that, compared with ZnO, ZC contains N element, indicating that ZC realized the adsorption of type I collagen

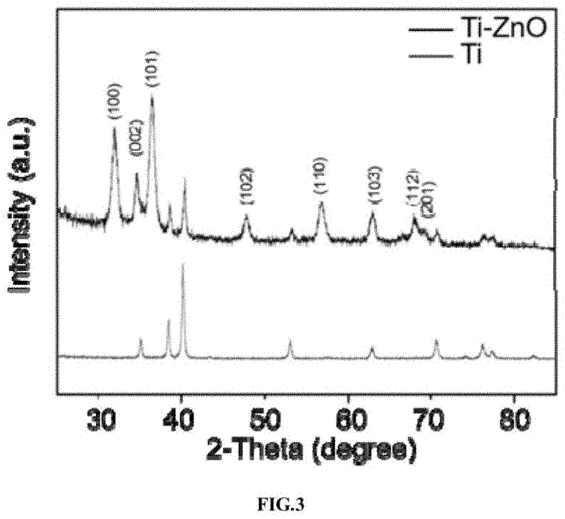

The crystal structures of initial Ti and Ti—ZnO samples were determined by X-ray diffraction (XRD, BrukerD8A A25 X), with the results shown in FIG. 3. It can be seen from FIG. 3 that, the characteristic absorption peak of ZnO can be found in Ti—ZnO.

Embodiment 3 Protein Adsorption Capacity

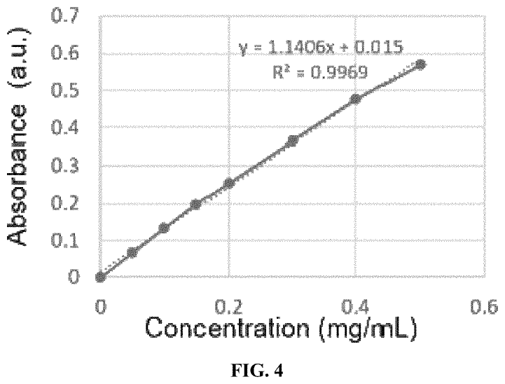

With bovine serum albumin (BSA) as the simulated protein, this embodiment utilized a BSA kit to detect the protein adsorption capacity of ZnO. ZnO of different mass (1 mg, 2 mg, 5 mg) was respectively placed in 1 mL BSA solution (in which the concentration of BSA was 5 mg/mL), and stirred at a speed of 75 r/min at 37° C. for 2 h, then washed twice centrifugally at a speed of 7000 r/min to get the supernatant. The resulting supernatant was diluted by 10 times, and placed into a 96-well plate. The standard curve of bovine serum albumin of known concentrations (as shown in FIG. 4) was used as the standard curve, and the absorbance at 562 nm was read with a microplate spectrophotometer.

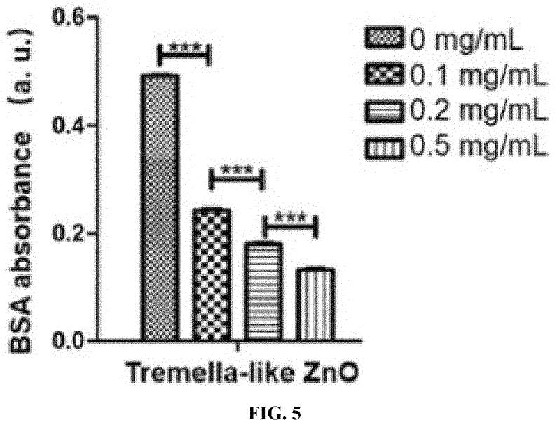

The results were shown in FIG. 5. FIG. 5 is a chart showing the adsorption results of BSA by different concentrations of zinc oxide, in which the concentration of ZnO is based on the concentration after dilution by 10 times. Where, the vertical ordinate indicates the absorbance of BSA, the lower the absorbance value, the more adsorbed by zinc oxide, indicating the better the adsorption capacity of zinc oxide. It can be seen from FIG. 5 that, the zinc oxide prepared in the present disclosure can adsorb BSA, and with the increase of the concentration of zinc oxide, the adsorption amount of BSA also increases.

Embodiment 4 Photocatalytic Performance

Ti—ZnO was placed in a certain concentration of Rhodamine B solution and shaken at a speed of 150 rpm. Two groups of experiment were set, in which one group was in dark, and the other group was irradiated with yellow light. The color changes of the Rhodamine B solution were observed after 12 h, 24 h, 36 h and 48 h, respectively. The supernatant was determined at different time points with an ultraviolet spectrophotometer to determine the OD values of the absorption peaks of Rhodamine B and calculate the degradation rate (R value) of Rhodamine B.

The calculation formula is: R=C₀−C/C₀, in which C is the OD values of the absorption peaks of Rhodamine B after stirring for 12 h, 24 h, 36 h and 48 h, and C0 is the initial OD value of the absorption peaks of Rhodamine B.

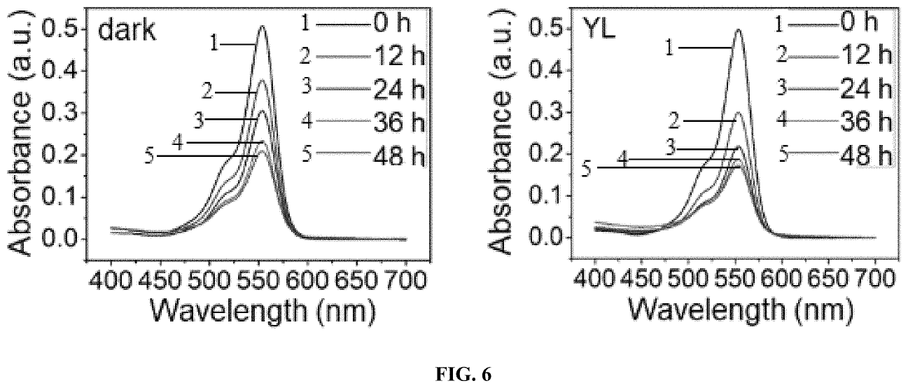

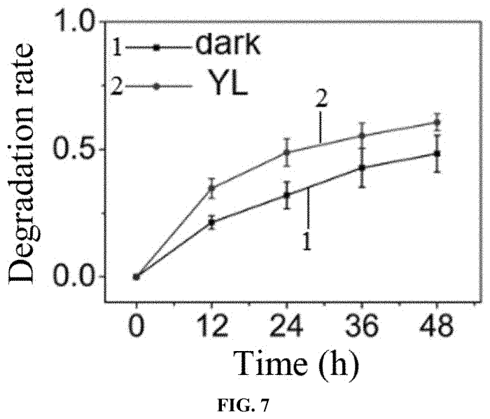

The results were shown in FIGS. 6-7. FIG. 6 shows the ultra-violet absorption spectra of Rhodamine B solution under dark and light conditions respectively for 0 h, 12 h, 24 h, 36 h and 48 h; FIG. 7 is a curve showing the changes of degradation rate of Rhodamine B under dark and light conditions respectively. It can be seen from FIGS. 6-7 that, as the extension of time, the degradation rate of Rhodamine B increases, and the degradation rate of Rhodamine B is higher under the irradiation of yellow light, indicating that the zinc oxide prepared in the present disclosure has an excellent photocatalytic performance.

Embodiment 5 Photothermal Conversion Ability

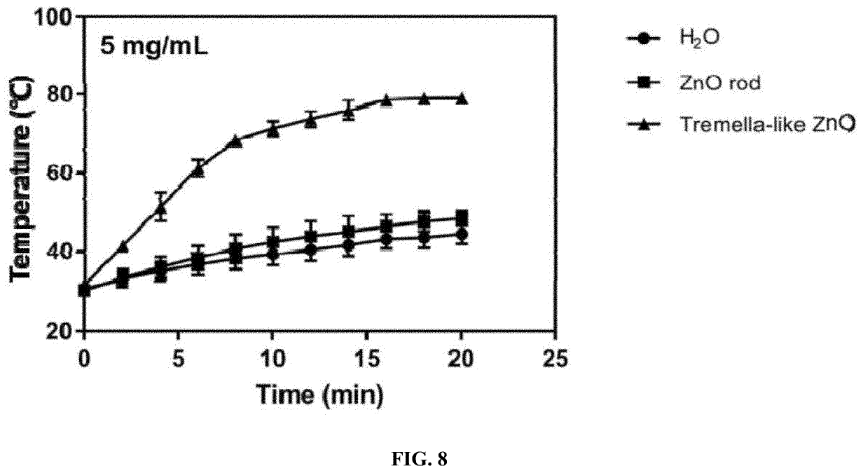

The Tremella-like zinc oxide (Tremella-like ZnO) prepared in embodiment 1 and conventional cylinder zinc oxide (ZnO rod) were dispersed in water, both of which were controlled at 5 mg/mL, and placed under the irradiation of near-infrared light (NIR, 808 nm) at room temperature. The effects of temperature rising were tested with water as the control, with the results shown in FIG. 8. FIG. 8 is a diagram showing the temperature changes of different kinds of zinc oxide dispersion under light condition. It can be seen from FIG. 8 that, compared with conventional cylinder zinc oxide, the Tremella-like zinc oxide prepared in the present disclosure has a stronger photothermal conversion ability, and the aqueous dispersion containing the Tremella-like zinc oxide of the present disclosure has a larger amplitude of temperature rising under the same conditions.

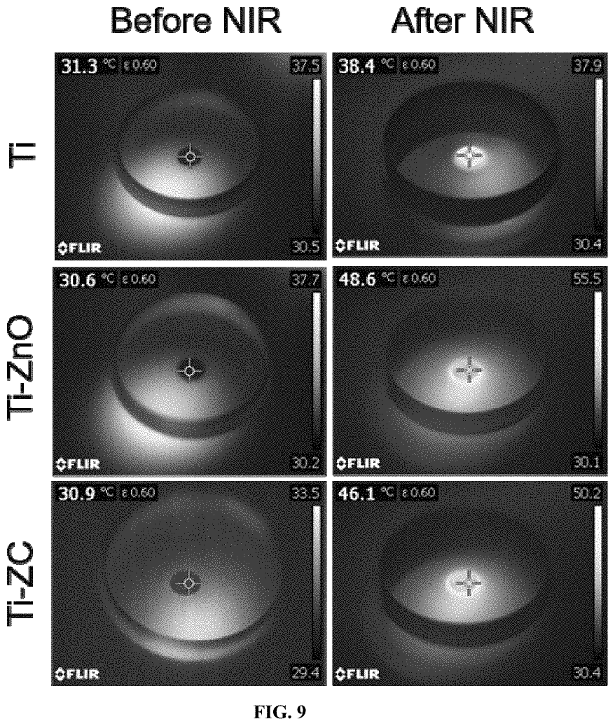

Ti (i.e., titanium sheets without any treatment), and Ti-ZC and Ti—ZnO prepared in embodiment 2 were irradiated under near-infrared light (NIR, 808 nm) for 2 min, and monitored with a FLIR A35 infrared thermal imager, with the results shown in FIG. 9. FIG. 9 shows a thermal imaging map of titanium samples through different treatments before and after NIR irradiation. It can be seen from FIG. 9 that, the temperature of Ti group rise to 38.4° C. after NIR irradiation, while the temperature of Ti—ZnO group rise to 48.6° C., and the temperature of Ti-ZC group rise to 46.1° C. The results show that, the zinc oxide prepared in the present disclosure has an excellent photothermal conversion ability.

Embodiment 6 In Vitro Antibacterial Ability

S. aureus, E. coli and S. mutans were selected to access the antibacterial ability of Ti samples. Before co-cultivation with bacteria, all the Ti samples were placed in 48-well plates, into which 500 μL bacterial solution (the concentration was 10⁷ CFU/mL) was added and cultivated at 150 rpm at 37° C. for 6 h (one plate was in dark, and the other plate was irradiated with yellow light). S. aureus and E. coli were cultivated in Luria-Bertani broth, while S. mutans was cultivated in brain heart infusion broth (BHI). The bacterial morphology was detected. All the bacterial specimen were fixed with 2.5% glutaraldehyde, dehydrated with ethyl alcohol gradiently (30, 50, 70, 80, 90, 100% v/v) and dried with a lyophilizer, and finally observed by SEM.

Where, Ti samples were Ti (i.e., initial titanium sheets), Ti—ZnO, and Ti-ZC, and a blank control group was set in which no Ti samples were added. The experiment groups in dark environment were labelled as Ctrl (blank control), Ti, Ti—ZnO, Ti-ZC, respectively; and the experiment groups in yellow light irradiation environment were labelled as YL (blank control), Ti-YL, Ti—ZnO-YL, Ti-ZC-YL, respectively.

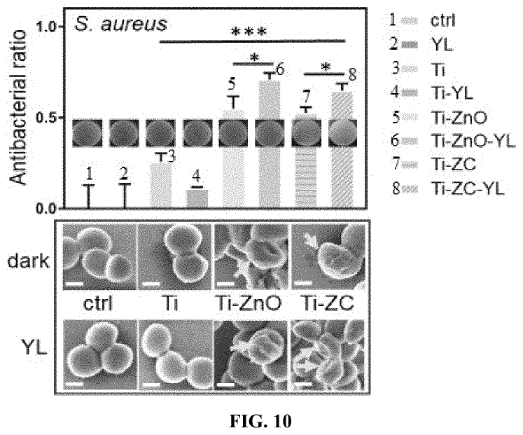

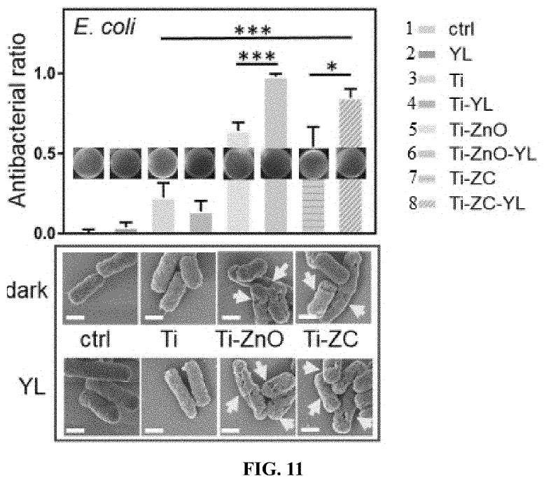

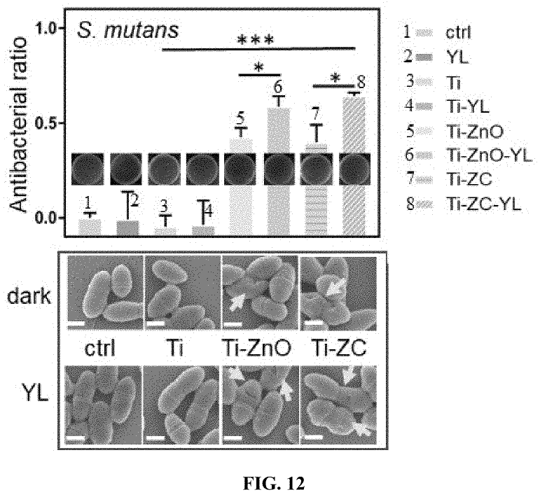

The results were shown in FIGS. 10-12. FIG. 10 shows the antibacterial ratio of Ti samples against S. aureus and the SEM view of the bacterium; FIG. 11 shows the antibacterial ratio of Ti samples against E. coli and the SEM view of the bacterium; and FIG. 12 shows the antibacterial ratio of Ti samples against S. mutans and the SEM view of the bacterium. In FIGS. 10-12, the scale of all the SEM views is 1 μm.

It can be seen from FIGS. 10-12 that, after cultivation of 6 h, the antibacterial efficiencies of Ti and Ti-YL groups are both lower than those of corresponding Ti—ZnO and Ti—ZnO-YL groups. The antibacterial ratios of Ti against S. aureus, E. coli and S. mutans were 25.71%, 22.38% and −0.04% respectively, while the antibacterial ratios of Ti—ZnO against S. aureus, E. coli and S. mutans were 54.94%, 64.1% and 42.41% respectively, demonstrating that the presence of ZnO in Ti—ZnO samples improves the antibacterial ability of Ti. In addition, when exposed to yellow light, the antibacterial ratio of Ti—ZnO was further improved significantly (S. aureus 70.87%, E. coli 97.9%, S. mutans 58.69%). This is related to the photocatalytic activity of ZnO produced under the irradiation of yellow light. ZnO can produce reactive oxygen species under light, which have certain killing effect on bacteria. After loading with Col-I, there was no significant effect on the antibacterial property (Ti-ZC), but when combined with yellow light, the antibacterial activity was improved (Ti-ZC-YL).

It can be seen from the SEM views of the microstructures of different bacterial groups as shown in FIGS. 10-12 that, the bacteria in the control group have normal structures, complete membranes and regular shapes; and the same complete morphologies are also observed in the bacteria of YL, Ti and Ti-YL groups. On the contrary, the bacteria in Ti—ZnO, Ti—ZnO-YL, Ti-ZC and Ti-ZC-YL groups are all wrinkled and irregular, and the cell membrane are broken to varying degrees (as indicated by arrows). The above results show that, the coating provided in the present disclosure has an excellent antibacterial property, and the antibacterial property may be improved significantly as the introduction of yellow light.

Embodiment 7 In Vitro Photothermal Capability

It has been reported in documents that proper thermal therapy can promote bone remodeling to some extent. The heating effects of different Ti samples on BMSCs (bone mesenchymal stem cells) under NIR conditions are tested in this embodiment.

In an environment of 37° C. and 5% CO₂, BMSCs were cultivated in DMEM medium containing 10% fetal calf serum. The third generation of BMSCs was used for subsequent cell detection. Ti samples were immersed in DMEM for 72 h to get impregnating liquid, which was filtered over a filter membrane of 0.22 μm before use. Where, the Ti samples were Ti and Ti-ZC.

24 h before the experiments, BMSCs were inoculated into 48-well plates. Then, 500 μL impregnating liquid was added into the plates and incubated for 24 h. Each well was irradiated with 808 nm laser (1.0 W/cm²), and the temperatures were recorded every 2 min, until the temperature of Ti-ZC group rose to about 40° C. (such a temperature is beneficial to osteogenesis), the irradiation was terminated. The temperature of each well was then monitored by an infrared thermal imaging camera to get an infrared image.

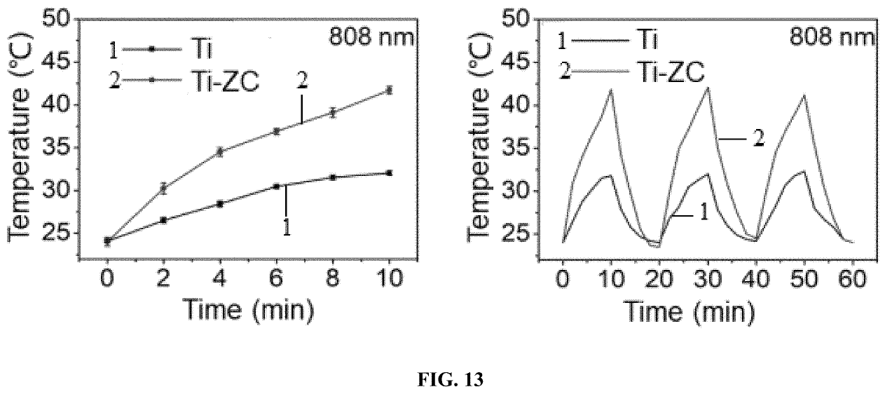

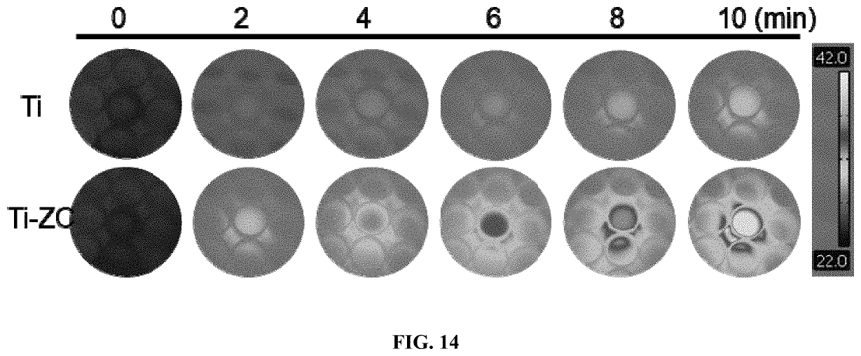

The results were shown in FIG. 13-FIG. 14. FIG. 13 is a diagram showing the temperature changes of cells treated with different impregnating liquids under NIR irradiation, in which the left panel of FIG. 13 is the temperature rising diagram of the cell groups treated with different impregnating liquids under NIR irradiation, and the right panel is the temperature changing diagram of the cells treated with different impregnating liquids in three cycles of NIR irradiation (when the temperature of Ti-ZC rose to about 40° C., the irradiation was terminated, and when decreased to normal temperature, the irradiation was initiated again, for 3 cycles in total). It can be found from FIG. 13 that, with the irradiation of NIR, the temperature of Ti-ZC group rises gradually, and the rising amplitude of temperature is significantly higher than that of Ti group; and after three cycles of NIR irradiation, the Ti-ZC groups show stable temperature switching effects, indicating that Ti-ZC has a good photostability.

FIG. 14 shows the infrared thermogram of cells treated with different impregnating liquids after continuous NIR irradiation for 10 min. In this experiment, the temperature change in Ti group was the minimum, and the temperature of Ti-ZC group rose to about 40° C. gradually. Therefore, Ti-ZC has a strong photothermal effect and good photothermal stability, which is a great candidate material for photothermal therapy.

Embodiment 8 Expression of Osteogenic Gene

In this embodiment, the expression level of runt-related transcription factor 2 (Runx2) and osteocalcin (OCN) of BMSCs in different treatment groups were tested. These two kinds of proteins have an important role in bone remodeling, and the upregulation of their expression level can promote the osteoblastic differentiation of cells. The specific steps are as below:

BMSCs were inoculated in 6-well plates (10⁵ cells in each well), and cultivated in different impregnating liquids (the method of preparing impregnating liquids was consistent with that in embodiment 6, which were Ti impregnating liquid and Ti-ZC impregnating liquid, respectively) for 7 days. One group was cultivated without near-infrared irradiation, and the other group was cultivated in a medium with near-infrared (1.0 W/cm²) irradiation for 10 min every day, where the medium was in an incubator at 37° C. and 5% CO₂, and replaced every 3 days. The gene expression of Runx2 and OCN were detected with related primers. The expression level of all the targeting genes was normalized to GAPDH.

After removing the medium carefully, the cells were fixed on ice with a paraformaldehyde solution (4%) for 15 min, and then washed with PBS. After then, the cells were detected with a BCIP/NBT ALP (alkaline phosphatase) color development kit (Beyotime Institute of Biotechnology) for 15 min. After washing with dd H₂O, the ALP staining level was recorded.

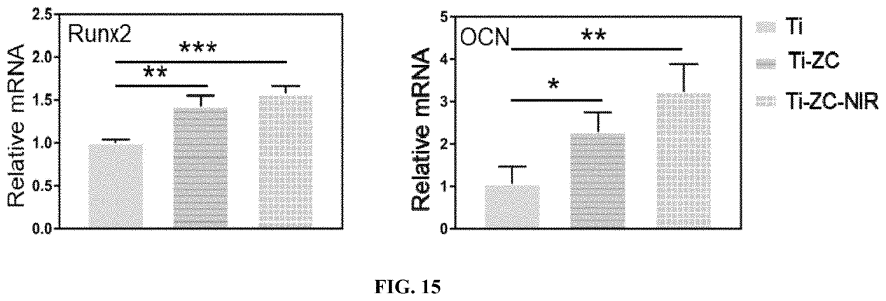

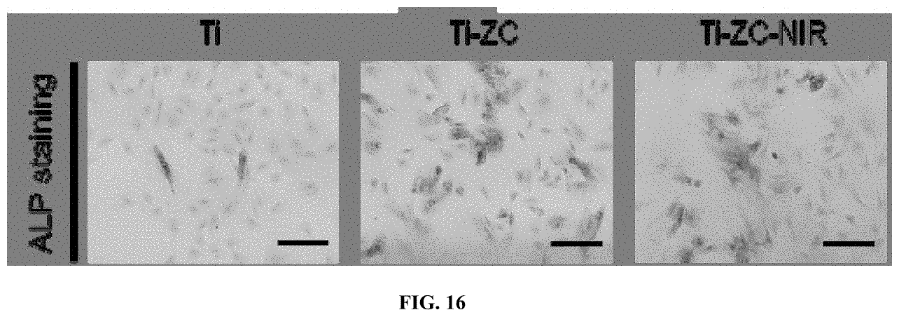

The results were shown in FIGS. 15-16. FIG. 15 shows the gene expression level of Runx2 and bone OCN of cells after being treated with different impregnating liquids; FIG. 16 shows the testing results of ALP staining level of cells after being treated with different impregnating liquids, where the scale is 200 μm. It can be seen from FIG. 15 that, after Ti-ZC and Ti-ZC-NIR treatment, the expression of cell Runx2 and OCN was increased, and NIR irradiation further up-regulates the expression of Runx2 and OCN in BMSCs. This is probably related to the excellent photothermal property of ZnO on the surface of Ti samples. The results of ALP staining (FIG. 16) also verify the above conclusions.

It is demonstrated from the above experimental results that the coating of the present disclosure has a photothermal effect, which can improve the activity of ALP and up-regulate the expression level of Runx2, OCN and other osteogenic genes, thus having an important role in promoting the osseointegration on the surface of implants.

The description of the above embodiments is only intended to assist in understanding the method and core concept of the present disclosure. It should be noted that several improvements and modifications can be made to the present disclosure by persons with ordinary skills in the art without deviating from the principle of the present disclosure, all of which also fall within the protection scope of claims of the present disclosure. Various modifications to these embodiments are apparent to technical personnel in the art. General principles defined herein can be realized in other embodiments without deviating from the spirit or scope of the present disclosure. Therefore, the present disclosure shall not be confined to these embodiments set forth herein, but shall conform to the widest scope consistent with the principle and novel features disclosed herein.

What is claimed is:

1. A preparation method of dual light-responsive zinc oxide, comprising the following steps:
(1) a soluble zinc salt, hexamethylenetetramine and water are mixed for a first hydrothermal reaction, and then the reaction material liquor is mixed with sodium citrate, hydroxypropyl methyl cellulose, photothermal conversion materials and lignin for a second hydrothermal reaction, to get hydrothermal products;
(2) the hydrothermal products are subjected to lyophilization and microwave irradiation successively to get the dual light-responsive zinc oxide.

2. The preparation method according to claim 1, wherein, the soluble zinc salt is zinc nitrate.

3. The preparation method according to claim 1, wherein, the photothermal conversion materials are one or more of activated carbon, gold rod and black phosphorus.

4. The preparation method according to claim 1, wherein, the dosage ratio of the soluble zinc salt, hexamethylenetetramine, water, sodium citrate, hydroxypropyl methyl cellulose, photothermal conversion materials and lignin is (1.487-1.488) g:(0.350-0.352) g:(95-110) mL:(0.138-0.142) g:(0.100-0.120) g:(0.025-0.035) g:(0.995-0.115) g.

5. The preparation method according to claim 2, wherein, the dosage ratio of the soluble zinc salt, hexamethylenetetramine, water, sodium citrate, hydroxypropyl methyl cellulose, photothermal conversion materials and lignin is (1.487-1.488) g:(0.350-0.352) g:(95-110) mL:(0.138-0.142) g:(0.100-0.120) g:(0.025-0.035) g:(0.995-0.115) g.

6. The preparation method according to claim 3, wherein, the dosage ratio of the soluble zinc salt, hexamethylenetetramine, water, sodium citrate, hydroxypropyl methyl cellulose, photothermal conversion materials and lignin is (1.487-1.488) g:(0.350-0.352) g:(95-110) mL:(0.138-0.142) g:(0.100-0.120) g:(0.025-0.035) g:(0.995-0.115) g.

7. The preparation method according to claim 1, wherein, for the first hydrothermal reaction, the temperature is 65-67° C., and the time is 14-16 min; for the second hydrothermal reaction, the temperature is 85-87° C., and the time is 10-12 h.

8. The preparation method according to claim 1, wherein, for the microwave irradiation, the power is above 800 W, and the time is more than 15 min.

9. A dual light-responsive zinc oxide prepared by the method of claim 1, wherein, the dual light-responsive zinc oxide has a Tremella-like microtopography; and the dual light-responsive zinc oxide can respond under the irradiation of yellow light and near-infrared light.

10. The dual light-responsive zinc oxide according to claim 9, wherein, the soluble zinc salt is zinc nitrate.

11. The dual light-responsive zinc oxide according to claim 9, wherein, the photothermal conversion materials are one or more of activated carbon, gold rod and black phosphorus.

12. The dual light-responsive zinc oxide according to claim 9, wherein, the dosage ratio of the soluble zinc salt, hexamethylenetetramine, water, sodium citrate, hydroxypropyl methyl cellulose, photothermal conversion materials and lignin is (1.487-1.488) g:(0.350-0.352) g:(95-110) mL:(0.138-0.142) g:(0.100-0.120) g:(0.025-0.035) g:(0.995-0.115) g.

13. The dual light-responsive zinc oxide according to claim 9, wherein, for the first hydrothermal reaction, the temperature is 65-67° C., and the time is 14-16 min; for the second hydrothermal reaction, the temperature is 85-87° C., and the time is 10-12 h.

14. The dual light-responsive zinc oxide according to claim 9, wherein, for the microwave irradiation, the power is above 800 W, and the time is more than 15 min.

15. A photosensitive coating with antibacterial/osteogenic properties, wherein, which is prepared from the dual light-responsive zinc oxide of claim 9.

16. The photosensitive coating according to claim 15, wherein, the coating is prepared by the following steps:
the dual light-responsive zinc oxide is dispersed in a solvent, the resulting suspension is coated on the surface of a substrate and dried to get the photosensitive coating with antibacterial/osteogenic properties; and the substrate is a surgical implant.

17. The photosensitive coating according to claim 15, wherein, the raw materials for preparing the coating further include type I collagen powder, and the mass ratio of the dual light-responsive zinc oxide to the type I collagen powder is 1:(1-5).

18. The photosensitive coating according to claim 16, wherein, the raw materials for preparing the coating further include type I collagen powder, and the mass ratio of the dual light-responsive zinc oxide to the type I collagen powder is 1:(1-5).
